(19) United States
(12) Patent Application Publication
(10) Pub. No.: US 2026/0209665 A1
(43) Pub. Date: Jul. 23, 2026

(54) TRANSWELL ADAPTER

(71) Applicant: HACETTEPE UNIVERSITESI REKTORLUK, Cankaya/Ankara (TR)

(72) Inventors: Ibrahim Cagatay KARAASLAN, Ankara (TR); Sema TUNCER, Ankara (TR)

(73) Assignee: HACETTEPE UNIVERSITESI REKTORLUK, Cankaya/Ankara (TR)

(21) Appl. No.: 19/142,250

(22) PCT Filed: Dec. 27, 2023
(86) PCT No.: PCT/TR2023/051743

§ 371 Date: Jun. 23, 2025

(30) Foreign Application Priority Data
Dec. 27, 2022 (TR) 2022/020709

(51) Int. Cl. C12M 1/12; C12M 1/00; C12M 1/32

(52) U.S. Cl. CPC C12M 25/04; C12M 23/12; C12M 23/26

(57) ABSTRACT

The present invention relates to a “transwell” adapter for facilitating cell culture to be performed in “transwells” used in three-dimensional air-liquid interface (ALI) cultures and increasing the yield.

### TECHNICAL FIELD

The present invention relates to a “transwell” adapter for facilitating cell culture to be performed in “transwells” (permeable membrane between equipment/wells wherein cells are cultured in co-culture applications) used in three-dimensional air-liquid interface (ALI) cultures and increasing the yield.

### BACKGROUND OF THE INVENTION

In the current technique, the cells adhere to the bottom surface of the plate as a result of inverting the lid and keeping it in the incubator after culturing on the bottom surface of the insert during co-culture application and this leads to a decrease in the number of cells that should adhere in the desired medium. In addition, this makes it difficult to adjust the cell-material suspension and may lead to problems in evenly distributing the medium over all cells. In the event that a higher volume is cultured, this may cause overflow of the medium from the surface of the insert and in the event that a smaller volume is cultured, serious problems may arise such as drying of the medium and/or insufficient nutrition of the cells. In the traditional approach in ALI culture system, only one surface of the “transwell” is often used for cell culturing. Since there is no commercial apparatus for 3D use and co-culture applications, researchers who want to use “transwell” can basically culture on both surfaces of the “transwell” in two ways. In the first approach, a low volume (one drop) of cell-medium suspension is added onto the inverted “transwell” membrane and the cells are expected to adhere to the membrane surface in this environment depending on the type of cell. In this method, the incubation time is extremely important and in case of long-term incubation, the cells may die by being short of medium upon the medium evaporates at 37° C. Short incubation periods make it difficult particularly for sensitive primary cells to adhere to the membrane surface. In the second approach, the “Transwell” is inverted and a chamber wherein the medium will remain for a short time is created by wrapping the outer surface of it is wrapped with a parafilm or duct tape. In addition to the fact that this chamber is not durable enough for the medium to remain for a long time, it also creates an open source for contamination caused by the parafilm and the duct tape used. Also, these materials are not biocompatible materials. Since they are in direct contact with the medium, they may also lead to a problem as a source of chemical contamination.

Therefore, a “transwell” adapter which will overcome the above-stated shortcomings is needed.

The United States patent document no. US2017183622, an application included in the state of the art, discloses a biocompatible membrane made by electrospinning a polymer and depositing living cells on both surfaces of the membrane. In each embodiment, the invention comprises a biocompatible membrane. The membrane is not harmful to living tissue, including specifically human cells, human lung cells, human brain cells, human astrocytes, human brain microvascular endothelial cells, human small airway epithelial cells, human lung microvascular endothelial cells, or other cells. Suitable membrane materials include but not limited to gelatin, collagen, polycaprolactone (PCL), polylactic acid (PLA), polyglycolic acid (PGA) and poly(DL-lactide-co-glycolide) (PLGA).

### SUMMARY OF THE INVENTION

An objective of the present invention is to realize a “transwell” adapter for facilitating cell culture to be performed in “transwells” used in three-dimensional air-liquid interface (ALI) cultures and increasing the yield.

### DETAILED DESCRIPTION OF THE INVENTION

“A Transwell Adapter” realized to fulfil the objective of the present invention is shown in the figures attached, in which:

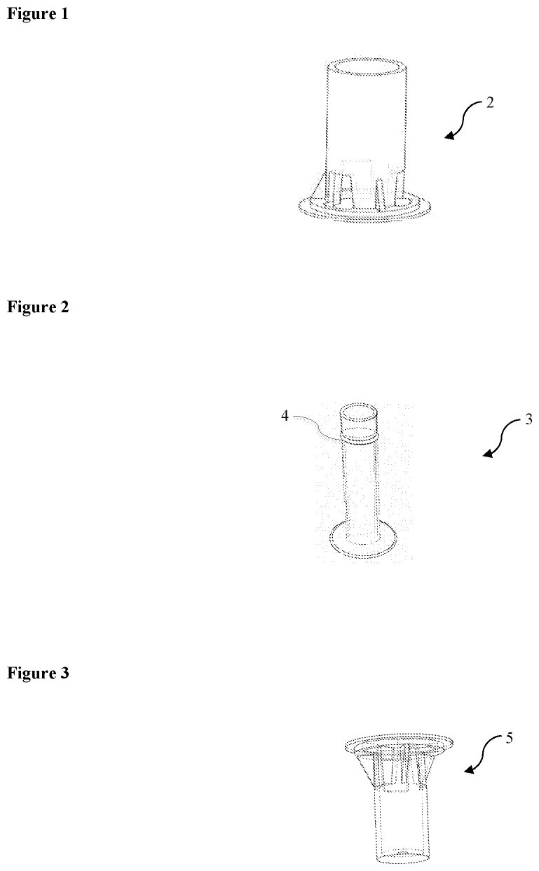

FIG. 1 is a view of the “transwell” in the inventive “transwell” adapter,

FIG. 2 is a view of the lower adapter in the inventive “transwell” adapter.

FIG. 3 is a view of the upper adapter in the inventive “transwell” adapter.

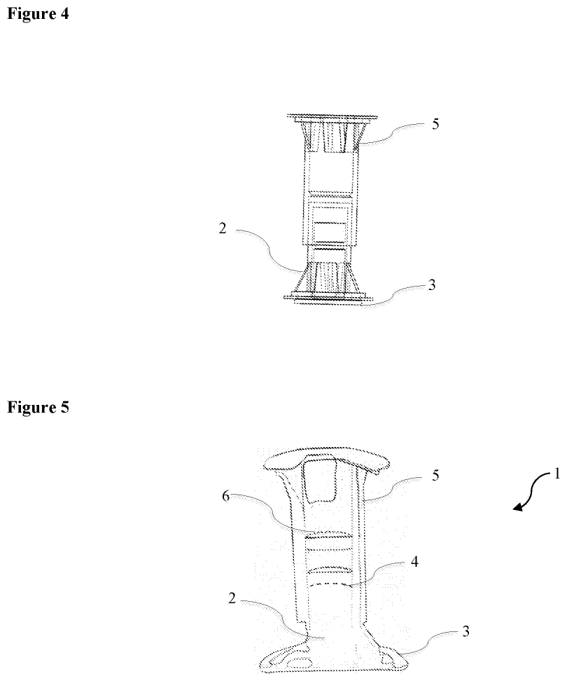

FIG. 4 is a view of the lower adapter, the upper adapter and the “transwell” fitted together in the inventive “transwell” adapter.

FIG. 5 is a cross-sectional view of the lower adapter, the upper adapter, the membrane and the “transwell” fitted together in the inventive “transwell” adapter.

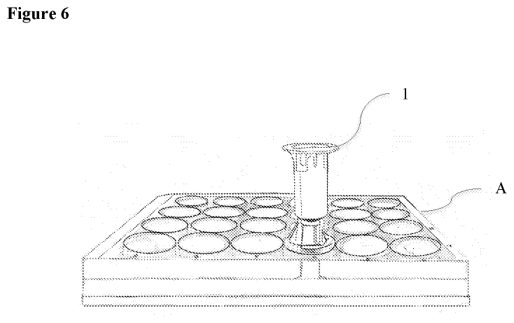

FIG. 6 is a view of the inventive “transwell” adapter inserted into a 24-well plate.

The components illustrated in the figures are individually numbered, where the numbers refer to the following:
1. Transwell adapter
2. Transwell
3. Lower adapter
4. Sealing element
5. Upper adapter
6. Membrane
A: 24-well plate

The inventive “transwell” adapter (1) for facilitating cell culture to be performed in three-dimensional air-liquid interface (ALI) cultures and increasing the yield, comprises
at least one “transwell” (2) which contains a hollow cylinder with one open end and one closed end, and a part conically expanding from the open end of the cylinder,
at least one lower adapter (3) which contains a solid cylinder closed at both ends and a plate integral with the cylinder-that enables the cylinder to stand upright on the ground-at one end of the cylinder and has at least one annular recess at the section close to the end of the cylinder where there is no plate;
at least one sealing element (4) which has a diameter suitable for insertion into the recessed section of the lower adapter (3) and is fitted into this recess;
at least one upper adapter (5) which contains a hollow cylinder open at both ends, and a part conically expanding from one of the open end of the cylinder; and
at least one membrane (6) which is placed on the outer surface of the closed cylinder end of the “transwell” (2).

The cylinder diameter of the lower adapter (3) included in the inventive “transwell” adapter (1) has a diameter suitable for tightly entering into the cylindrical structure of the “transwell” (2). The lower adapter (3) is inserted into the “transwell” (2) by means of shrink fit by being advanced from the open end of the “transwell” (2) towards the cylindrical part of the “transwell” (2) from its end where there is no plate. The lower adaptor (3) is used to prevent the medium from passing to the lower part when culturing on the reverse surface of the membrane (6).

The sealing element (4) included in the inventive “transwell” adapter (1) has a diameter suitable for insertion into the recess in the lower adapter (3). The sealing element (4) has a ring-shaped flexible structure. The sealing element (4) is configured to ensure that the “transwell” (2) and the lower adapter (3) are hermetically fitted together.

The upper adapter (5) included in the inventive “transwell” adapter (1) is attached to the closed end of the “transwell” (2), that is attached to the lower adapter (3) standing on the floor by means of its plate, from its non-tapered part. The cylinder diameter of the non-tapered part of the upper adapter (5) has a diameter suitable for being tightly inserted from the outside into the cylinder section of the non-tapered part of the “transwell” (2). The upper adapter (5) is configured to form a medium reservoir capable of retaining the fluid during cell adhesion and incubation after cell culture is performed on the reverse surface of the membrane (6).

The membrane (6) included in the inventive “transwell” adapter (1) is the place where cell culture is performed in co-culture applications. The membrane (6) has a nanofibre structure synthesised by electrospinning of two biodegradable polymers in the form of PLLA (poly-L-lactic acid) and PCL (polylactic acid). The membrane (6) is inserted on the outer surface of the closed cylindrical shape of the “transwell” (2) by being adhered via a biocompatible adhesive. The biocompatible adhesive used in one embodiment of the invention is Kwik-sil (silicone elastomer, WPI). The membrane (6) has the shape of a round disc with a diameter almost equal to the diameter of the cylinder of the “transwell” (2).

The novel “transwell” adapter (1) is prepared by placing the lower adapter (3) on a floor from the plate section, inserting the sealing element (4) into the recess in the lower adapter (3), inserting the open end of the “transwell” (2) to the part of the lower adapter (3) wherein the sealing element (4) is inserted, and then adhering the biodegradable membrane (6) to the outer surface of the closed part of the “transwell” (2) via a biocompatible adhesive, and finally inserting the upper adapter (5) to the “transwell” (2) from its non-tapered part so as to include the part of the “transwell” (2) where the membrane (6) is adhered.

The process steps followed for the use of the said “transwell” adapter (1) in the process of cell culturing in air-liquid interface (ALI) cultures are as follows: The lower adapter (3) with the sealing element (4) is placed into the 24-well plate (A). The “transwell” (2) is placed on the lower adapter (3) and the upper adapter (5) is placed on the “transwell” (2). The membrane (6) is located on the “transwell” (2).

A cover frame is placed around the 24-well plate (A). Cell culture is performed in the “transwell” adapter (1). After the cell culture, the cover of the 24-well plate (A) is closed. The plate (A) is placed in the incubator. After 24 hours, the plate (A) is removed from the incubator and the medium included on thereof is removed. The upper adapter (5) is removed from the “transwell” adapter (1). The “transwell” (2) is separated from the lower adapter (3). The “transwell” (2) is hold by means of forceps and placed in the 24-well plate (A). A medium is added into the upper and lower chamber (apical-basal) of the plate (A). The cover of the plate (A) is closed and placed in the incubator again.

With the inventive “transwell” adapter (1), a desired cell/medium suspension ratio can be achieved during reverse culture of cells, the desired number and time of cells can be cultured and it is ensured that the medium does not flow and/or dry out during incubation. On the other hand, in applications of ALI cell culture, a practical system design whereby both sides of the “transwell” membranes can be cultured is achieved. In addition, integration of “transwells” and biodegradable membrane in applications of ALI culture paves the way for the use of tissue grafts which can be used in possible airway damages.

Within these basic concepts; it is possible to develop various embodiments of the inventive “Transwell Adapter (1)”; the invention cannot be limited to examples disclosed herein and it is essentially according to claims.

1. A “transwell” adapter (1) for facilitating cell culture to be performed in three-dimensional air-liquid interface (ALI) cultures and increasing the yield; comprising
at least one “transwell” (2) which contains a hollow cylinder with one open end and one closed end, and a part conically expanding from the open end of the cylinder;
and characterized by
at least one lower adapter (3) which contains a solid cylinder closed at both ends and a plate integral with the cylinder—that enables the cylinder to stand upright on the ground-at one end of the cylinder and has at least one annular recess at the section close to the end of the cylinder where there is no plate;
at least one sealing element (4) which has a diameter suitable for insertion into the recessed section of the lower adapter (3) and is fitted into this recess;
at least one upper adapter (5) which contains a hollow cylinder open at both ends, and a part conically expanding from one of the open end of the cylinder; and
at least one membrane (6) which is placed on the outer surface of the closed cylinder end of the “transwell” (2).

2. A “transwell” adapter (1) according to claim 1; characterized by the lower adapter (3) with a cylinder diameter which has a diameter suitable for tightly entering into the cylindrical structure of the “transwell” (2).

3. A “transwell” adapter (1) according to claim 1; characterized by the lower adapter (3) which is inserted into the “transwell” (2) by means of shrink fit by being advanced from the open end of the “transwell” (2) towards the cylindrical part of the “transwell” (2) from its end where there is no plate.

4. A “transwell” adapter (1) according to claim 1; characterized by the lower adapter (3) which is used to prevent the medium from passing to the lower part when culturing on the reverse surface of the membrane (6).

5. A “transwell” adapter (1) according to claim 1; characterized by the sealing element (4) which has a diameter suitable for insertion into the recess in the lower adapter (3).

6. A “transwell” adapter (1) according to claim 1; characterized by the sealing element (4) which has a ring-shaped flexible structure.

7. A “transwell” adapter (1) according to claim 1; characterized by the sealing element (4) which is configured to ensure that the “transwell” (2) and the lower adapter (3) are hermetically fitted together.

8. A “transwell” adapter (1) according to claim 1; characterized by the upper adapter (5) which is attached to the closed end of the “transwell” (2), that is attached to the lower adapter (3) standing on the floor by means of its plate, from its non-tapered part.

9. A “transwell” adapter (1) according to claim 1; characterized by the upper adapter (5) wherein the cylinder diameter of its non-tapered part has a diameter suitable for being tightly inserted from the outside into the cylinder section of the non-tapered part of the “transwell” (2).

10. A “transwell” adapter (1) according to claim 1; characterized by the upper adapter (5) which is configured to form a medium reservoir capable of retaining the fluid during cell adhesion and incubation after cell culture is performed on the reverse surface of the membrane (6).

11. A “transwell” adapter (1) according to claim 1; characterized by the membrane (6) which is the place where cell culture is performed in co-culture applications.

12. A “transwell” adapter (1) according to claim 1; characterized by the membrane (6) which has a nanofibre structure synthesised by electrospinning of two biodegradable polymers in the form of PLLA and PCL.

13. A “transwell” adapter (1) according to claim 1; characterized by the membrane (6) which is inserted on the outer surface of the closed cylindrical shape of the “transwell” (2) by being adhered via a biocompatible adhesive.

14. A “transwell” adapter (1) according to claim 1; characterized by the membrane (6) which has the shape of a round disc with a diameter almost equal to the diameter of the cylinder of the “transwell” (2).

15. A “transwell” adapter (1) according to claim 1; which is prepared by placing the lower adapter (3) on a floor from the plate section, inserting the sealing element (4) into the recess in the lower adapter (3), inserting the open end of the “transwell” (2) to the part of the lower adapter (3) wherein the sealing element (4) is inserted, and then adhering the biodegradable membrane (6) to the outer surface of the closed part of the “transwell” (2) via a biocompatible adhesive, and finally inserting the upper adapter (5) to the “transwell” (2) from its non-tapered part so as to include the part of the “transwell” (2) where the membrane (6) is adhered.
